(19) United States
(12) Patent Application Publication
(10) Pub. No.: US 2006/0275351 A1
(43) Pub. Date: Dec. 7, 2006

(54) TOPICAL OLIGOPEPTIDE DELIVERY SYSTEM

(76) Inventors: Fatemeh Mohammadi, Hauppauge, NY (US); Vasile Ionita-Manzatu, Old Bethpage, NY (US); Andrew J. Bevacqua, East Setauket, NY (US); Julius R. Zecchino, Closter, NJ (US); Anna Czarnota, Huntington, NY (US); Mirela Ionita-Manzatu, Old Bethpage, NY (US)

Correspondence Address: THE ESTEE LAUDER COS, INC, 125 PINELAWN ROAD, MELVILLE, NY, 11747, US

(21) Appl. No.: 11/421,834

(22) Filed: Jun. 2, 2006

Related U.S. Application Data
(60) Provisional application No. 60/687,738, filed on Jun. 6, 2005.

(51) Int. Cl. A61F 13/02; A61N 1/30; A61K 38/08

(52) U.S. Cl. 424/448; 514/17; 604/20

(57) ABSTRACT

The invention relates to a delivery system for biologically active oligopeptides. The delivery system comprises an electrochemical cell and a skin-beneficial amount of one or more oligopeptides. In a preferred embodiment the electrochemical cell and the peptide are contained in a dermal patch. The invention also relates to a topically acceptable hydrogel that comprises a hydrophilic polymer, an aqueous or aqueous alcoholic carrier, at least one skin-beneficial oligopeptide, and a salt, wherein at least a portion of the carrier is a structured water.

This application claims the benefit of Provisional application 60/687,738, filed Jun. 6, 2005.

### FIELD OF THE INVENTION

The invention relates to the field of topical delivery of cosmetic or pharmaceutical actives. More specifically, it relates to a delivery system for biologically active oligopeptides.

### BACKGROUND OF THE INVENTION

The delivery of pharmaceutical compositions for the treatment of a variety of conditions using electrical current to enhance delivery is well known, and the skin is a frequent site for such mechanisms of delivery. The techniques of iontophoresis and electroporation routinely rely on the skin as the pathway for delivery of therapeutic compositions that are intended ultimately for systemic distribution. Although historically these procedures have required complicated electrical equipment to achieve its end, modern technology has greatly simplified matters. A particularly convenient means for utilizing an electrically driven delivery of active materials is a dermal patch comprising power cell components. Typically, such patches contain at least two electrodes, one positive and one negative, and a configuration for completing the circuit between the two electrodes, so as to generate the desired electrical current when in contact with the skin.

Patches of this type have been used, or recommended for use, with a variety of different types of active materials, for treatment of both skin-related and non-skin-related disorders. They have not yet, however, to the best of Applicants' knowledge, been utilized in the delivery of oligopeptides. Applicants have now determined that application of oligopeptides to the skin in combination with an electrochemically generated current provides effective delivery to the skin cells in a gentle and efficient manner.

### SUMMARY OF THE INVENTION

The invention relates to a delivery system for a skin-beneficial oligopeptide comprising an electrochemical cell and a skin-beneficial amount of the oligopeptide. In a preferred embodiment the cell and the peptide a contained in a dermal patch. The invention also relates to a hydrogel comprising a topically acceptable hydrogel comprising a hydrophilic polymer, an aqueous or aqueous alcoholic carrier, at least one skin-beneficial oligopeptide, and a salt, wherein at least a portion of the carrier is a structured water.

### BRIEF DESCRIPTION OF THE DRAWINGS

FIG. 1 is a graph demonstrating the effect of the delivery system on moisturization of the skin over time;

FIG. 2 is a graph demonstrating the effect of the delivery system on lines and wrinkles in the skin over time as measured by the change in Integrated Optical Density;

FIG. 3 is a graph demonstrating the effect of the delivery system on Transepidermal Water Loss of skin over time;

FIG. 4 is a graph demonstrating the effect of the delivery system on skin over time as measured by Replica Analysis;

FIG. 5 is a graph demonstrating the effect of the delivery system on skin over time as measured by panelist self evaluation;

FIG. 6 is a graph demonstrating the effect of the delivery system on skin over time as measured by Clinical Assessment; and

FIG. 7 is a graph demonstrating the effect of the delivery system on skin on skin firmness over time.

### DETAILED DESCRIPTION OF THE INVENTION

Throughout this specification, the terms “comprise”, “comprises”, “comprising”, “have”, “has” and “having” and the like shall consistently mean that a collection of objects is not limited to those objects specifically recited.

Oligopeptides, as defined herein, are short peptides comprising between two and 20 amino acid residues. Preferably, the oligopeptides of the invention comprise between three and 10 amino acid residues. The oligopeptides of the invention can be any that have beneficial effect on skin cells. Examples of useful skin benefits include whitening, free-radical scavenging, anti-aging, stimulation of collagen synthesis, moisturizing, antimicrobial, anti-inflammatory, or anti-irritant. In a preferred embodiment, oligopeptides are used for treating or preventing the effects of photo- or chronoaging of the skin.

The delivery system of the invention comprises applying the oligopeptide to the skin in combination with a gentle microcurrent. The source of the microcurrent can be any number of devices and/or vehicles that are capable of generating a current in situ on the skin; for purposes of the present invention, a microcurrent is defined as a current that generates no more than 3 volts on the skin, preferably less than 1.5 volts. The delivery of therapeutic oligopeptides by way of these microcurrent-generating devices or vehicles differs from delivery by more typical iontophoretic or electroporetic devices, in that the current is generated on the skin itself, by interaction of the components of the delivery system, rather than requiring an external source of electricity.

In one embodiment, the microcurrent is delivered by way of a dermal patch. As an example of such a delivery system, U.S. Pat. No. 5,652,043 discloses a compact electrochemical cell adaptable to such purpose, and US Application Nos. 2004/167461, 2004/267189, and WO03/35166 disclose the application of that type of electrochemical cell to a dermal patch. The contents of each of these documents is incorporated herein by reference in their entirety. Such patches are also available commercially from Power Paper (21 Yagia Kapayim Kiriyat Arie, Petach Tikiva, Israel 49130).

In brief, the electrochemical cell generates a current in a manner similar to conventional batteries, with an electron donor and an electron acceptor separated by an electrolyte (a solution of ions that conducts electricity). The anode (positive pole) and the cathode (negative pole) comprise compounds capable of exchanging electrons, for example, manganese dioxide for the anode and zinc for the cathode. The ability of the cathode and anode to generate a current is enabled by the presence of an electrolytic solution which connects them ionically, allowing the necessary exchange of electrons.

The preferred patch of the invention comprises an open cell, i.e., one in which the electrolytic portion of the electrochemical cell is not sealed in a sheathing film, thereby preventing the accumulation of gases inside the cells. The structure of the electrolytic portion of the cell typically comprises a porous substrate, such as paper, plastic, cellulose or cloth, saturated with an aqueous solution containing at least one hygroscopic material, at least one water soluble electroactive material, and at least one water soluble polymer with adhesive properties. Examples of hygroscopic materials include but are not limited to calcium chloride, calcium bromide, potassium biphosphate, or potassium acetate. The water soluble electroactive material (the electrolyte per se) may be any topically acceptable conductive material, such as zinc chloride, zinc bromide, zinc fluoride, potassium hydroxide and sulfuric acid. The water soluble adhesive polymer may be any topically acceptable polymer such as PVA, polyacrylamide, polyacrylic acid, PVP, polyethylene oxide, agar, starch, or derivatives thereof. In some embodiments, one material may serve a dual purpose, e.g. zinc bromide or chloride can serve as both the hygroscopic material and the electroactive material, while a starch such as dextran or dextranesulfate can function as the hygroscopic material as well as the water soluble polymer.

The preferred patch also comprises negative and positive poles, each of which is a mixture of an insoluble electroactive powder and an aqueous solution such as is described for the electrolyte. For the poles, the electroactive materials of the electrolyte and the electroactive material of the poles must be the same, although the hygroscopic and polymeric materials may be different. Suitable electroactive powder combinations for the negative and positive poles include, but are not limited to, MgO₂—Zn; SO₂—Zn; Cd—NiO₂; or I—NiO₂. The electrochemical cell is constructed by saturating a porous substrate with the aqueous solution as described above; depositing a layer of negative pole mixture on one side of the porous substrate; depositing a layer of positive pole mixture on the other side of the porous substrate. Thus, there are three layers in the basic cell, thin and flexible. The aqueous solution and pole mixtures may be applied to the substrate by any method, but printing is preferred. Just about any known printing technology can be used in which the aqueous solution and pole mixtures are treated as inks being printed to a substrate. The substrate can be any shape and the “inks” can be laid down in any pattern. Each layer of pole mixture applied to the substrate may be further covered by a conductive layer (i.e. graphite, carbon cloth). Terminals (graphite or metal) are connected to either each pole layer or their associated conductive layers. The terminals provide points of attachment for an electric load. The terminals are preferably printed onto the substrate. A portion of the terminals may rise above the substrate. An additional adhesive backing may be supplied on one side of the cell. A protective lamina may be applied to a portion of the cell surface. Two or more of the basic cells just described may be stacked to produce additive power output. The basic cell may be as thin as 0.5 mm, and produce 1.5 to 3 volts in the microamp range.

A particularly preferred cell is disclosed in WO03/35166, which describes a topical device capable of supplying an electric current below the surface of the skin. The source of the electric current may be any electric current generator capable of supplying direct current at the specified voltages and amperage; however, the preferred power source is a miniature thin, flexible, electrochemical cell, and most preferably an open, liquid state electrochemical cell as described in the cited reference.

The patch device uses a variation of the basic electrochemical cell. In the basic cell, one electrode is located at the bottom of the stacked layers and the other at the top. If this basic cell is placed on the skin, then the bottom electrode is adjacent to the skin. To make the top electrode contact the skin the top electrode is fashioned in an extended shape that reaches from the top pole layer of the cell to the skin. To prevent short circuiting, an insulating layer may be provided to isolate the top electrode from the lower pole layer and electrode. With this configuration, both electrodes are adjacent the skin when the electrochemical cell is laid on the skin. There is a potential, then, for current to flow between the electrodes by passing through the skin.

For convenience, the electrochemical cell is housed in a flexible patch body. A portion of the base of the body is covered with a biocompatible adhesive for attaching the patch to the skin. The patch is used in conjunction with dermatological and pharmaceutical compounds contained in a conductive fluid, typically comprising water, or an alcoholic/aqueous solution, at least one salt (for example, sodium or potassium chloride) or any other charged agent, and optionally a buffering medium. The conductive fluid may be, for example, an electrically conductive hydrogel, and preferably an adhesive hydrogel, suitable for use as a skin contact. A hydrogel is a gel prepared with hydrophilic polymers, and these materials are well known in the art, frequently being used as part of biomedical electrodes, such as are described in U.S. Pat. Nos. 6,631,294 and 6,845,272, the contents of which are incorporated herein by reference. Examples of hydrophilic polymers useful for the preparation of hydrogels are polyacrylate, polymethacrylate, polyacrylamide, poly(vinyl alcohol), poly(ethylene oxide), poly(ethylene imine), carboxy-methylcellulose, methylcellulose, poly(acrylamide sulphonic acid), polyacrylonitrile, poly(vinyl-pyrrolidone), agar, dextran, dextrin, carrageenan, xanthan, and guar. The preferred hydrogels are cationic acrylates and may be, for example, preferably made from acrylic esters of quatenary chlorides and/or sulfates or acrylic amides of quaternary chlorides; polymers of this type are disclosed in U.S. Pat. No. 5,800,685, incorporated herein by reference. The hydrophilic polymers will generally constitute from about 1 to about 70%, preferably about 5 to about 60%, more preferably about 10 to about 50%, by weight of the hydrogel.

The conductive fluid may be applied to each electrode, prior to applying the patch to the skin or the conductive fluid may be applied to two locations on the skin for contact with the electrodes after the patch is applied to the skin. The conductive fluid at one location should not contact the fluid at the other, or the electric current will not pass into skin. In a preferred embodiment, the conductive fluid is supplied in a retainer that allows precise positioning of the conductive fluid.

Such a patch can conveniently be adapted to the delivery of skin-beneficial oligopeptides to the skin. For example, the oligopeptides of interest may conveniently be incorporated into a hydrogel that serves as the conductive fluid in the patch. Any oligopeptide that produces a skin benefit may be so incorporated. A number of oligopeptides having skin benefits are known in the art. For example, it is known that certain fragments of larger, skin-beneficial proteins, such as collagen or fibrin, can be used to promote collagen or fibrin synthesis when applied topically. Additional examples of useful oligopeptides for the purpose of the present invention is the group of oligopeptides disclosed in U.S. Pat. No. 6,620,419, the contents of which are incorporated herein by reference. The oligopeptides disclosed therein have the formula R.₁—X-Thr-Thr-Lys-(AA)ₙ-Y and salts thereof wherein X is a basic amino acid of D or L orientation, such as lysine, arginine, histidine, ornithine, citrulline, sarcosine, statine), (AA)ₙ represents a chain of n amino acids, natural or synthetic, wherein n is an integer from 0 to 5, R.₁ is H or a fatty acid chain of 2 to 22 carbons, hydroxylated or not, saturated or not, linear or branched, sulfurated or not, cyclic or not, or a biotin group, or a protective group of the urethane type used in peptide synthesis such as the groups benzyloxycarbonyl (Z), terbutyloxycarbonyl (tBoc), fluorenylmethyloxycarbonyl (Fmoc), allyloxycarbonyl (Alloc), and Y═OR.₂ or NR.₂R.₃ wherein R.₂ and/or R.₃ are a hydrogen atom or an aliphatic or aromatic chain of 1 to 22 carbons, hydroxylated or not, saturated or not, linear or branched, sulfurated or not, cyclic or not. These oligopeptides are disclosed as having anti-aging and moisturizing properties. Particularly preferred peptides are palmitoyl pentapeptides. Such a pentapeptide is commercially available under the trade name Matrixyl, from the company, Sederma.

Similarly, U.S. Pat. No. 6,372,717 (incorporated herein by reference) discloses peptides containing the sequence Tyr-Arg, particularly lipophilic derivatives of such peptides, as having a soothing effect on the skin, thereby reducing irritation and sensitivity. The lipophilic peptides have the formula R1-L-Tyr-L-Arg-R2 in which R1 is a group R3-C═O wherein R3 is an alkyl chain of C1 to C20, linear or branched, saturated or unsaturated, hydroxylated or not, or with R3 being an aryl, aryl-alkyl, or alkyloxy, group, and in which R2 is a group O—R4 wherein R4 is an alkyl chain of C1 to C20, or R2 is an NH2 or NHX or NXX group wherein X is an alkyl chain of C1 to C4. Such peptides may also be effectively applied with microcurrents in accordance with the present invention.

EP 1180524 discloses a group of catecholamine inhibiting oligopeptides that have the effect of reducing the appearance of lines and wrinkles on the skin. The contents of this document are incorporated by reference. These peptides are derived from the carboxy end of protein SNAP-25. Any of these peptides may be useful in connection with the patch of the invention; however, one particularly useful peptide has the formula Glu-Glu-Met-Gln-Arg-Arg. More specifically, a peptide of this type is acetyl hexapeptide3, also known commercially as Argireline®, manufactured by Lipotec, and available from Centerchem (Norwalk, Conn.).

Additional examples of skin-beneficial oligopeptides include oligopeptides obtained by the biotransformation of native proteins from the seeds of Hibiscus esculents L. (okra), commercially available as a complex in Myoxinol LS 9736 from Cognis. It is primarily composed of low molecular weight oligopeptides.

The oligopeptides utilized in the electrochemical cells of the invention will vary in the final concentrations used, but generally will be employed in the amounts normally recommended for their use when applied directly to the skin without the aid of the electrochemical cell, or may be used in slightly lower amounts, because of the efficacy of delivery accomplished in the use of the cell. One or more oligopeptides can be used in a conductive fluid, and oligopeptides exhibiting different types of activities can also be combined in a single conductive fluid. Overall, the oligopeptides will ordinarily be incorporated into the conductive fluid in amounts of from about 0.001 to about 50%, preferably from about 0.01 to about 30%, more preferably about 0.1 to about 20%, by weight of the conductive fluid composition.

The conductive fluid, as noted above, will also contain components such as water or a water/alcohol mix. Alcohols used are preferably polyhydric alcohols, such as glycols, such as pentylene glycol, or glycerol, which may also have a beneficial humectant effect. The water employed can be any water that is capable of acting as a conductor, but in a preferred embodiment, the water employed is a structured water, i.e., I water, S water, or a combination of the two, as described, for example, in RO 88053 [S-type water], and RO 88054 [-type water], and U.S. Pat. Nos. 5,846,397 and 6,139,855, the contents of each of which are incorporated herein by reference. As a general rule, the clustering of ions in structure water(s) enhances the biological properties or modifies the biochemical behavior of a particular material, when used in the presence of the water, as is described in U.S. Pat. Nos. 5,846,397 and 6,139,855, the contents of which are incorporated herein by reference. Therefore, the combination of the chosen oligopeptide(s) with one or both of either I water or S water can further enhance the efficacy of the oligopeptide on the skin. The water or water/alcohol component ordinarily will comprise from about 1 to about 65% by weight of the hydrogel, preferably between about 2 and about 55%, and more preferably from about 4 to about 50%.

In addition to the oligopeptide active, it may also be desirable to add one or more skin-benefit components. Examples of such skin benefit agents include, but are not limited to, astringents, such as clove oil, menthol, camphor, eucalyptus oil, eugenol, menthyl lactate, witch hazel distillate; antioxidants or free-radical scavengers, such as ascorbic acid, its fatty esters and phosphates, tocopherol and its derivatives, N-acetyl cysteine, sorbic acid and lipoic acid; anti-acne agents, such as salicylic acid and benzoyl peroxide; antimicrobial or antifungal agents such as caprylyl glycol, triclosan, phenoxyethanol, erythromycin, tolnaftate, nystatin or clortrimazole; chelating agents, such as EDTA; topical analgesics, such as benzocaine, lidocaine or procaine; anti-aging/anti-wrinkle agents, such as retinoids or hydroxy acids; skin lightening agents, such as licorice, ascorbyl phosphates, hydroquinone or kojic acid), skin-conditioning agents (e.g., humectants, including miscellaneous and occlusive), antiirritants, such as cola, bisabolol, aloe vera or panthenol, anti-inflammatories, such as hydrocortisone, clobetasol, dexamethasone, prednisone, acetyl salicylic acid, glycyrrhizic acid or glycyrrhetic acid; anti-cellulite agents, such as caffeine and other xanthines; humectants, such as alkylene polyols or hyaluronic acid; emollients, such as oily esters or petrolatum; sun protecting agents (organic or inorganic), such as avobenzone, oxybenzone, octylmethoxycinnamate, titanium dioxide or zinc oxide; exfoliating agents (chemical or physical), such as N-acetyl glucosamine, mannose phosphate, hydroxy acids, lactobionic acid, peach kernels, or sea salts; self-tanning agents, such as dihydroxyacetone; and biologically active peptides, such as palmitoyl pentapeptide or argireline. These supplemental skin benefit agents will be used in the amounts normally known to be effective for that active when used for the intended purpose.

The delivery of the oligopeptide to the skin is accomplished by contacting the electrochemical cell with the skin, and substantially simultaneously contacting the skin with the conductive fluid; preferably the conductive fluid and cell are applied to the skin together, as part of a single delivery device, such as the dermal patch. In employing a patch, the patch is positioned on the skin where the skin-beneficial activity is required, and allowed to remain in place for a several minutes, typically five minutes to thirty minutes, and then removed. Depending on the intended end use, the delivery system is applied on an as-needed basis (for example, to reduce sensitivity) or chronically (for example, for treatment of the signs of aging, such as lines, wrinkles and skin atrophy, or for enhancing moisturization of the skin). Application will be performed from about once per week to about 4 or 5 times daily, preferably from about 3 times a week to about 3 times daily, most preferably about once or twice per day. Chronic application will be understood to mean a period of topical application that may be over the lifetime of the user, preferably for a period of at least about one month, more preferably from about three months to about twenty years, more preferably from about six months to about ten years, more preferably still from about one year to about five years. Once the conductive fluid is applied, electric current begins to flow. Positive ions accumulate under the anode and electrons under the cathode (as in any electrochemical cell). Once sufficient accumulation has occurred, the positively (negatively) charged species in the dermatological or pharmaceutical compound of the conductive fluid will be driven away from the anode (cathode) and into the skin.

The patch may be any shape and the electrodes may be any size and shape to match the size and shape of the surface to which it is applied, for example, under the eye, around the corner of the eye, above the lip, on the forehead, or for full facial coverage.

The invention will be further illustrated by the following non-limiting examples:

### EXAMPLE 1

A hydrogel comprising an oligopeptide is prepared as follows:

1. 86% of the gel consists of a commercially available hydrogel (First Water, Marlborough, Wilts., UK) comprising poly(2-acrylamido-2-methylpropane-sulfonate sodium salt co PEG400 diacrylate), glycerol, and potassium chloride salt

2. 14% of this gel consists of acetyl hexapeptide-3 (Argireline®: Glu-Glu-Meth-Glu-Arg-Arg) and Structured Waters (I and S).

| | |
|---|---|
| I Water | 36.35% |
| S Water | 59.40% |
| Hexapeptide | 0.50% |
| Pentylene Glycol | 3.00% |
| Phenoxyethanol | 0.75% |

The hydrogel is then incorporated as the conductive fluid of a dermal patch as described in US application nos. 2004/167461 and. 2004/267189. Such a patch is used in the clinical testing procedure described in Example 2.

### EXAMPLE 2

The study is composed of twenty-four (24) women who satisfied all the requirements itemized in the list of inclusion and exclusion criteria. The subjects ranged in age from forty-one (41) to sixty-nine (69), and were Fitzpatrick Skin Types I, II, and III. Six (6) subjects had Fitzpatrick Skin Type I, fourteen (14) subjects had Fitzpatrick Skin Type II, and four (4) subjects had Fitzpatrick Skin Type III. All subjects participating in the study had at least moderate wrinkles in the canthus region. Six (6) subjects possessed moderate wrinkles, fifteen (15) possessed deep wrinkles, and three (3) possessed extremely deep wrinkles. Participants were instructed not to use any other topical agents other than the Estee Lauder Patch for the duration of the study. Subjects were instructed to maintain their daily cleansing routine for the duration of the study.

The panelists were instructed to apply the patch on clean dry skin, to peel off the protective backing, to place the patch at least ¼ inch from the eyes, ensure the patch adheres to the face, leave it in place for 20 minutes and then remove it and discard. On Day 1 of the study, baseline measurements were taken. The investigator then applied Estee Lauder Patch to the right and left canthus area of each panelist. After 20 minutes, the patch was removed, and measurements were repeated. On Day 2 through Day 5, and at Weeks 2, 3, and 4, pre-treatment measurements were taken, followed by patch application. After 20 minutes, the patches were removed and measurements repeated. Weeks 5 and 6 were a regression period during which no patches were applied and only pre-treatment measurements were taken. The subjects acclimated in an environmental room at 40% relative humidity and 70 degrees F. for 20 minutes as the first step. Moisturization measurements were taken first, followed by photographs, TEWL, replicas, clinical and self assessment and ballistometer.

This controlled study consisted of 6 weeks total testing time. The test site was the canthus area. Measurements were taken on both the right and left sides of the face. The women refrained from using any treatment products on the test sites except for the test product provided. Skin evaluations were carried out at baseline, before patch application (pre-treatment), and immediately after patch removal at each visit. Panelists' skin was evaluated for skin moisturization, lines and wrinkles (via photography and self-assessment), transepidermal water loss, and skin firmness.

Skin moisturization is measured via the Nova Meter DPM 9003 (NOVA Technology Corporation, Portsmouth, N.H.). The Nova measures skin moisturization as a function of increased skin surface water content. The instrument measures an output proportional to the skin's electrical capacitance in the Mhz. frequency range. Data acquisition is software controlled. The difference in electrical capacitance before and after treatment is calculated. The higher the skin water content, the higher the electrical capacitance and hence, the more moisturized the skin.

Reduction of lines & wrinkles after product use is assessed and documented with close up photography. Photos of the right and left canthus are taken with a Fuji S2 digital camera. Panelists heads are placed in a head rest to insure reproducibility of positioning. The camera is positioned at a ratio of 1:7 and an F stop of 22. Photos are evaluated via an image analysis program, Optimas 6.51, comparing before and after product use. Lines and wrinkles are assessed by examining changes in the Integrated Optical Density (IOD) before and after product use. IOD is equal to [(255-grey value)×area]. A decrease in IOD represents a decrease in fine lines and wrinkles and vice-versa.

Transepidermal water loss is measured with a DermaLab® Evaporimeter (Cortex Technology, Denmark). The subjects are in a relaxed inclined position and they are not allowed to converse or get excited. Transepidermal water loss is recorded automatically and set at a 45 second total measurement time with a 15 second data acquisition period.

The subjects acclimate in an environmental room at 40% relative humidity and 70 degrees F. for 15-20 minutes. Measurements of TEWL are taken in three separate locations approximately 1 cm. apart in a row.

Lines and wrinkles are evaluated by a replica collection technique, followed by digital image analysis (Corcuff, P., Leveque, J. L., Skin Surface Replica Image Analysis of Furrows and Wrinkles, Handbook of Non-Invasive Methods and the Skin, CRC Press, Inc., Boca, Raton, Fla., 1995, 89-96.), as well as by a clinical and self-assessment.

Clinical evaluations were conducted by the trained co-investigator using a 10-point analog scale. The co-investigator was trained and qualified by an outside consultant, J. Close Associates. The purpose of the training was to identify and quantify the characteristics of skin parameters using human judges who have been specifically trained to evaluate objectively. A trained evaluator has an extensive perceptual vocabulary, draws from a common frame of reference, has experience in scale usage, and uses standardized evaluation techniques. For lines and wrinkles, a standard lexicon and references for that specific parameter were used for evaluation. The investigator did not refer back to the baseline scoring. Self evaluations were also performed by each panelist using the same 10-point analog scale. The subjects were trained in scale usage and were provided a common frame of reference.

The 10-point scale ranging from 0 for no lines and wrinkles, to 10 for extremely deep lines and wrinkles, was employed. The panelists were instructed not to refer back to the baseline scoring.

Skin firmness is assessed with the Ballistometer on the canthus area on both sides of the face. The ballistometer is an instrument that assesses the dynamic properties of the skin through the measurement of the rebound of a hard object on the surface of the skin. It measures skin elasticity by dropping a very light weight (1-5 grams) pendulum on the skin surface and measuring the rebound pattern of the pendulum via a computer. Once the probe hits the surface of the skin, the kinetic energy of the falling object is stored inside the skin, and is subsequently released to make the probe rebound at a smaller height than the initial starting position. To characterize the interaction between the pendulum and the skin, the differences in the amplitude of the first rebound are analyzed.

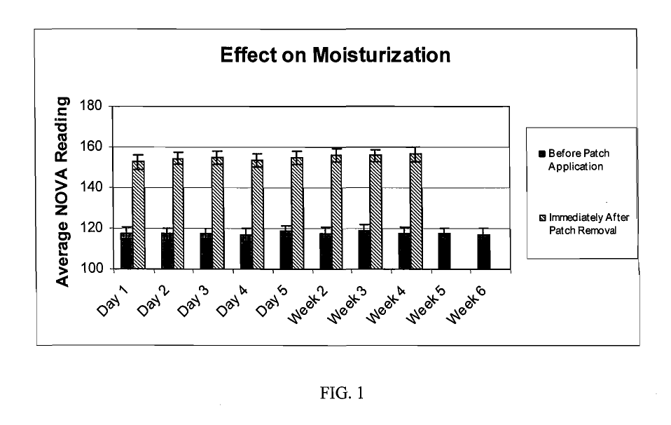

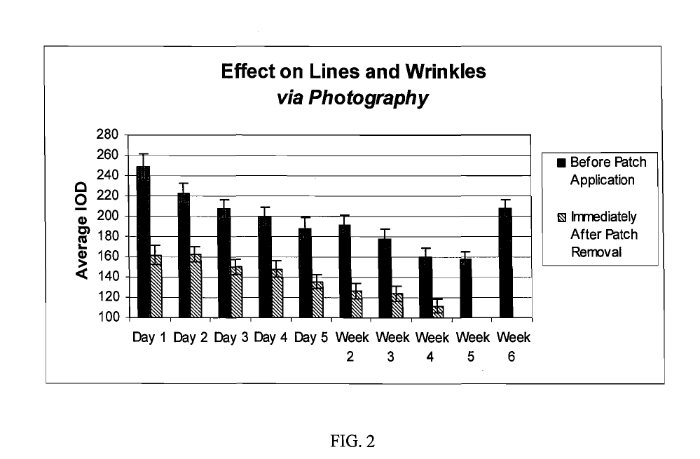

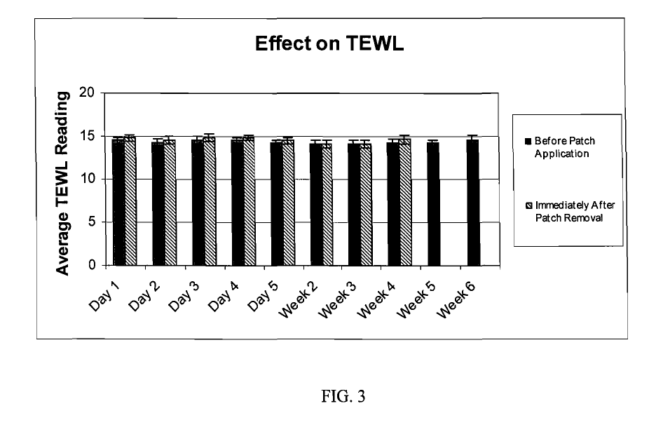

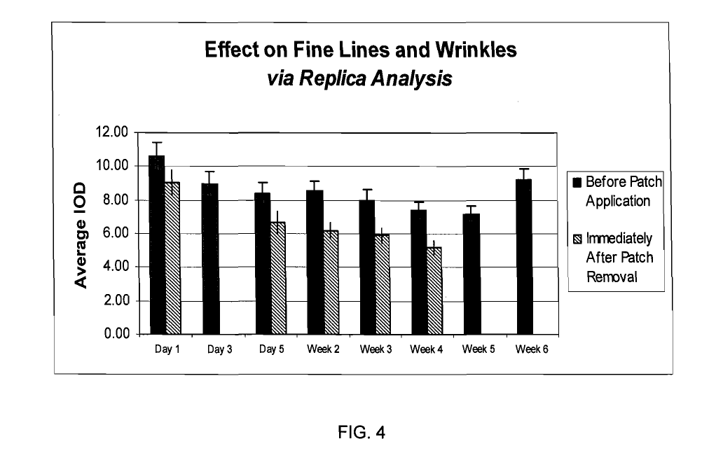

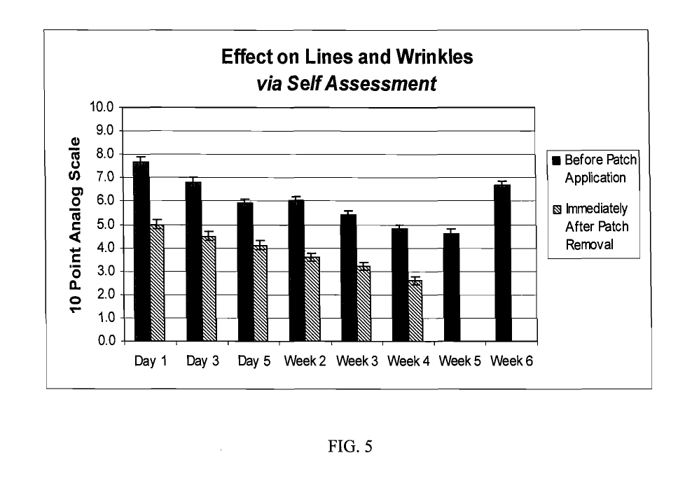

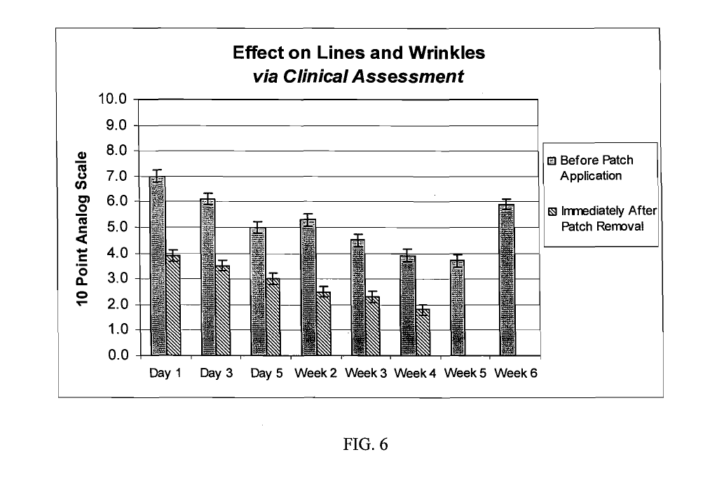

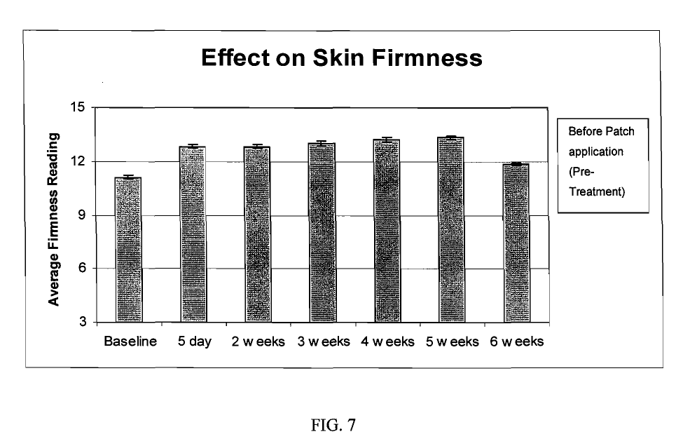

Statistics: The statistical significance of the data was analyzed through a two-sample paired student's t-test provided in Microsoft Excel. A two-tailed probability table was used to determine significance of the data. Pre-treatment data on Day 2-5 and Week 2-6 was compared to baseline, and compared to data immediately after patch removal on Day 1-5 and Week 2-4 was also compared to the baseline. The results are shown graphically in FIGS. 1-7.

### SUMMARY

The following were demonstrated immediately after one patch application, as compared to baseline:
30% improvement in skin moisturization
35% reduction in lines & wrinkles via photography
No change in TEWL, therefore, skin barrier was not disrupted
15% reduction in lines & wrinkles via replicas
27% reduction in lines & wrinkles via self assessment
31% reduction in lines & wrinkles via clinical assessment

The following were demonstrated 24 hours after one patch application (Pre-Treatment Day 2) as compared to baseline:
10% reduction in lines & wrinkles via photography

The following were demonstrated after eight patch applications, as compared to baseline:
37% reduction in lines & wrinkles via photography
No change in TEWL, therefore, skin barrier was not disrupted
33% reduction in lines & wrinkles via replicas
31% reduction in lines & wrinkles via self assessment
33% reduction in lines & wrinkles via clinical assessment
20% improvement in skin firmness

After a regression in which no patch was applied, all parameters began to return to baseline.

What we claim is:

1. A delivery system for a skin-beneficial oligopeptide comprising an electrochemical cell and a skin-beneficial amount of the oligopeptide.

2. The system of claim 1 in which the electrochemical cell is contained on a dermal patch.

3. The system of claim 1 in which the system comprises a conductive fluid.

4. The system of claim 3 in which the oligopeptide is contained in the conductive fluid.

5. The system of claim 4 in which the fluid is a hydrogel.

6. The system of claim 5 in which the hydrogel comprises a hydrophilic polymer selected from the group consisting of polyacrylate, polymethacrylate, polyacrylamide, poly(vinyl alcohol), poly(ethylene oxide), poly(ethylene imine), carboxy-methylcellulose, methylcellulose, poly(acrylamide sulphonic acid), polyacrylonitrile, poly(vinyl-pyrrolidone), agar, dextran, dextrin, carrageenan, xanthan, and guar, or mixtures thereof.

7. The system of claim 5 in which the hydrogel comprises at least one structured water.

8. The system of claim 7 in which the hydrogel comprises a combination of I and S structured waters.

9. The system of claim 1 in which the oligopeptide is selected from the group consisting of pentapeptides and hexapeptides.

10. The system of claim 9 in which the oligopeptide is selected from palmitoyl pentapeptides and acetyl hexapeptides.

11. The system of claim 10 in which the oligopeptide is acetyl hexapeptide 3.

12. A dermal patch oligopeptide delivery system comprising a porous substrate, an electrochemical cell, and a hydrogel containing a skin-beneficial oligopeptide.

13. The system of claim 12 in which the hydrogel contains a hydrophilic polymer, the oligopeptide, an aqueous or aqueous alcoholic carrier and a salt.

14. The system of claim 13 in which the carrier comprises at least one structured water.

15. The system of claim 14 which comprises at a combination of I and S structured water.

16. The system of claim 15 in which the oligopeptide is selected from pentapeptides and hexapeptides.

17. The system of claim 16 in which the oligopeptide is a palmitoyl pentapeptide or an acetyl hexapeptide/

18. The system of claim 17 in which the oligopeptide is acetyl hexapeptide 3.

19. A topically acceptable hydrogel comprising a hydrophilic polymer, an aqueous or aqueous alcoholic carrier, at least one skin-beneficial oligopeptide, and a salt, wherein at least a portion of the carrier is a structured water.
